(19) United States
(12) Patent Application Publication
(10) Pub. No.: US 2005/0124965 A1
(43) Pub. Date: Jun. 9, 2005

(54) Phosphatase inhibitor sample collection system

(75) Inventor: Bruce C. Haywood, Franklin Lakes, NJ (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 10/998,682

(22) Filed: Nov. 29, 2004

Related U.S. Application Data
(60) Provisional application No. 60/527,790, filed on Dec. 8, 2003.

(51) Int. Cl. A61M 31/00; A61K 31/727

(52) U.S. Cl. 604/500; 514/56

(57) ABSTRACT

A collection container and a method for collecting a biological sample, particularly whole blood, includes at least one stabilizing agent in an amount effective to stabilize and inhibit protein degradation and/or fragmentation. The stabilizing agent is able to stabilize proteases in the biological sample, particularly at the point of collection, by inhibiting protein degradation and/or fragmentation in the sample when the sample is stored. The stabilizing agent comprises or consists of one or more protease inhibitors.

### FIELD OF THE INVENTION

The present invention is directed to a method and device for collecting and stabilizing a biological sample, particularly a whole blood sample, directly from a patient. More specifically, the present invention relates to sample collection containers having a stabilizing additive contained therein for stabilizing proteins immediately on collection of a biological sample and for inhibiting protein modifications during storage thereof.

### BACKGROUND OF THE INVENTION

The study of proteomics has recently increased significantly. Proteomics may encompass many meanings, but it involves looking at proteins, whether individually or, more typically, as patterns. For example, researchers are interested in the protein profiles that may be reflective of certain disease states, e.g., the profile of a healthy individual vs. the profile of a diseased individual may show differences that can be used as future indicators of disease states. As known in the art, mass spectrometry is a key tool used to look at such profiles. One challenge in such protein study is the many modifications a protein goes through in its lifetime, which are broadly called post-translational modifications. Given that the state of a protein changes over time, it is difficult to ensure that a profile of an individual will be consistent over time. Thus, a profile believed to be indicative of a disease state may only be valid for specific conditions, and thus not repeatable on a basis sufficient to serve as a diagnostic tool. Thus, devices and/or processes capable of addressing this variability would be desirable.

### SUMMARY OF THE INVENTION

The most common mechanism of communication between cells involves the release of signaling molecules, such as hormones, neurotransmitters, and growth factors, from one cell type that interact with and activate specific receptor proteins on the surface of target cells. The activated receptor then generates an intracellular signal that ultimately couples to specific functional processes in cells to produce a physiological response. Studies of the signal transduction pathways that couple receptor activation to these physiological responses represent one of the most active and important research areas in modern biology. Signal transduction studies are fundamental to disease research, drug discovery and development, and diagnostics. The reversible attachment of phosphate to serine, threonine and tyrosine residues of cellular proteins is a control mechanism that plays a key role in most if not all signal transduction pathways. Two types of enzymes control the extent and direction of phosphorylation of a particular cell protein:
Protein kinases add phosphate to the proteins (phosphorylation)
Protein phosphatases remove the phosphate. (dephosphorylation)
   These pathway effects continue to be active after biological samples are collected. Without understanding of these variable ex-vivo modifications, phosphate removal, in particular, can confound or impair research results. Protein phosphatases are classified based on their substrate specificity, dependence on metal ions, and sensitivity to inhibitory agents. A class of chemicals, protein phosphatase inhibitors, is commonly used to limit removal of phosphate groups. (Protein phosphates inhibitors are also used to treat diseases).

There are hundreds of inhibitors available through chemical suppliers and some even provide inhibitor cocktails with anywhere from two to five inhibitors pre-mixed. It is unfortunate, that by the time most of these inhibitors are applied, that the much of the activity being studied is “unnatural” or ex vivo artifact. For certain studies, it is important to be able to understand the state of cells in a manner that is closely representative of the in vivo physiology. For this reason, there is value in regulating dephosphorylation as close to “time zero” of specimen excision or extraction as possible.

The invention includes a diverse range of collection devices that contain one or more pre-loaded protein phosphatase inhibitors, such that when the specimen contacts the collection device it immediately comes in contact with the inhibitor and the dephosphorylation activity is regulated.

### BRIEF DESCRIPTION OF THE DRAWINGS

FIG. 1 is a perspective view of a typical blood collection tube.

FIG. 2 is a perspective view of a test plate.

FIG. 3a is a perspective view of a sample collection assembly, while FIG. 3b is a sectional view of the sample collection assembly.

FIG. 4 is a longitudinal sectional view of a syringe.

FIG. 5 is a longitudinal sectional view of another embodiment of a syringe.

FIG. 6a is a side view of a catheter assembly, while FIG. 6b is a partial side view of the catheter.

FIG. 7 is a perspective view of a pipette.

FIG. 8 is a perspective view illustrating a blood collecting bag.

### DETAILED DESCRIPTION OF THE INVENTION

While this invention is satisfied by embodiments in many different forms, there will herein be described in detail preferred embodiments of the invention, with the understanding that the present disclosure is to be considered as exemplary of the principles of the invention and is not intended to limit the invention to the embodiments illustrated and described. Numerous variations may be made by persons skilled in the art without departure from the spirit of the invention. The scope of the invention will be measured by the appended claims and their equivalents.

The present invention is directed to methods and devices for stabilizing proteins in a biological sample to better enable downstream analysis. More particularly, the present invention is directed to methods and devices for inhibiting the phosphorylation cascade in a biological sample during storage. According to the present invention, the device comprises a container containing an amount of a stabilizing agent comprising a phosphatase inhibitor for mixing with a biological sample immediately on collection of the sample. Also according to the present invention, the method comprises providing a sample collection container containing a stabilizing agent in an amount sufficient to prevent or inhibit triggering of one or more phosphorylation cascades by inhibiting the phosphatase triggers and adding to the container a biological sample.

Although it is possible to use the present invention with any protein-containing biological sample, preferably the biological sample is any body fluid withdrawn from a patient. Most preferably, the biological sample is whole blood or a component thereof. Examples of other biological samples include cell-containing compositions such as red blood cell concentrates, platelet concentrates, leukocyte concentrates, plasma, serum, urine, bone marrow aspirates, cerebral spinal fluid, tissue, cells, feces, saliva and oral secretions, nasal secretions, lymphatic fluid and the like.

The sample collection system of the present invention can encompass any collection device including, but not limited to, tubes such as test tubes and centrifuge tubes; closed system blood collection devices, such as collection bags; syringes, especially pre-filled syringes; catheters, such as central lines; microtiter and other multi-well plates; arrays; tubing; laboratory vessels such as flasks, spinner flasks, roller bottles, vials, microscope slides, microscope slide assemblies, coverslips, films and porous substrates and assemblies; pipettes and pipette tips, etc.; tissue and other biological sample collection containers; and any other container suitable for holding a biological sample, as well as containers and elements involved in transferring samples. In one aspect of the invention, a sample collection tube having a separating member (e.g., a mechanical separating element, a gel or a filter mechanism) for separating blood components is used. In such aspect, the interior of the tube and/or the exterior of the separating member may be treated with the stabilizing agent. According to the present invention, the collection device contains a stabilizing agent for stabilizing the biological sample.

Plastic or glass is often used to manufacture the collection device used in the present invention. Some preferred materials used to manufacture the collection device include polypropylene, polyethylene, polyethyleneterephthalate, polystyrene, polycarbonate and cellulosics. More expensive plastics such as polytetrafluoroethylene and other fluorinated polymers may also be used. In addition to the materials mentioned above, examples of other suitable materials for the collection devices used in the present invention include polyolefins, polyamides, polyesters, silicones, polyurethanes, epoxies, acrylics, polyacrylates, polysulfones, polymethacrylates, PEEK, polyimide and fluoropolymers such as PTFE Teflong, FEP Teflon®, Tefzel®, poly(vinylidene fluoride), PVDF and perfluoroalkoxy resins. Glass products including silica glass are also used to manufacture the collection devices. One exemplary glass product is PYREX® (available from Corning Glass, Corning, N.Y.). Ceramic collection devices can be used according to embodiments of the invention. Cellulosic products such as paper and reinforced paper containers can also be used to form collection devices according to the invention.

The stabilizing agent of the invention comprises one or more phosphatase inhibitors able to inhibit phosphorylation activity and the associated modification or, destruction of proteins during storage of a biological sample. The agent stabilizes the biological sample, such as a blood sample, to produce a stable composition that inhibits or prevents modification, degradation and/or fragmentation of proteins present in the biological sample. In accordance with one embodiment of the present invention, the collection device is pre-treated with the stabilizing agent, preferably by the manufacturer, and is packaged in a ready-to-use form. Typically, the packaged collection device is sterile and is also packaged in sterile packaging materials.

The present invention could be used by pharmaceutical companies, biotechnology companies, contract research organizations, university researchers, research hospitals and any institution and individual who is interested in studying proteins. The present invention would enable researchers to conveniently and readily protect and process protein samples for downstream analysis. The collection device according to the present invention would serve as a front-end sample collection device aiding analytical objectives including, but not limited to the following: protein banking, protein identification and characterization, protein expression, protein quantitation, protein-protein interactions, development of protein function assays, protein target finding and validation, predictive toxicology, determination of drug action, drug validation, 3-D protein structural analysis and computer modeling. Clinical uses are also contemplated.

Preferably, the stabilizing agent comprises or consists of at least one phosphatase inhibitor. Suitable examples include inhibitors of serine or threonine phosphatases (e.g., the PPP or PPM families) and/or tyrosine phosphatases (the PTP family). For example, inhibitors of PP1 phosphatase include calyculin A, nodularin, NIPP-1, microcystin LR, tautomycin, okadaic acid, and cantharidin. Inhibitors of PP2A include calyculin A, microcystin LR, okadaic acid, fostriecin, tautomycin, cantharidin, endothall, and nodularin. Inhibitors of PP2B include cyclosporin A, FK 506/immunophilin complexes, cypermethrin, deltamethrin, and fenvalerate. Inhibitors of PTP include bpV(phen), dephostatin, mpV(pic) DMHV, and sodium orthovanadate. Phosphatases and inhibitors therefore are known in the art, and are commercially available, e.g., from Calbiochem of San Diego, Calif., USA.

Combinations of phosphatase inhibitors, commonly referred to as “cocktails” by commercial suppliers of such inhibitors, may also be used as the stabilizing agent. Such “cocktails” are generally advantageous in that they provide stabilization for a range of proteins of interest; therefore, a stabilizing agent containing more than two phosphatase inhibitors is generally desirable.

In addition, it may be desirable to include protease inhibitors with the phosphatase inhibitor, to further promote protein stability. Examples include inhibitors of proteases such as serine proteases, cysteine proteases, aspartic proteases, metalloproteases, thiol proteases, exopeptidases and the like. Of these, serine and cysteine protease inhibitors are of particular interest, with metalloprotease and aspartic inhibitors also being significant. Non-limiting examples of serine protease inhibitors include antipain, aprotinin, chymostatin, elastatinal, phenylmethylsulfonyl fluoride (PMSF), APMSF, TLCK, TPCK, leupeptin and soybean trypsin inhibitor. Inhibitors of cysteine proteases include, for example, IAA (indoleacetic acid) and E-64. Suitable examples of aspartic protease inhibitors include pepstatin and VdLPFFVdL. Non-limiting examples of inhibitors of metalloproteases include EDTA, as well as 1,10-phenanthroline and phosphoramodon. Inhibitors of exopeptidases include, for example, amastatin, bestatin, diprotin A and diprotin B. Additional suitable examples of protease inhibitors include alpha-2-macroglobulin, soybean or lima bean trypsin inhibitor, pancreatic protease inhibitor, egg white ovostatin and egg white cystatin.

The stabilizing agent may be in any suitable form including, but not limited to, a solution, suspension or other liquid, a pellet, a tablet, a capsule, a spray-dried material, a freeze-dried material, a powder, a particle, a gel, crystals, substrate-bound additive, buffered matrix, or a lyophilized material. Because the half-life of many inhibitors is short, the stabilizing agent is preferably introduced into the collection device in such a form so as to optimize the shelf life of the inhibitor. Lyophilization appears to be particularly useful in that it provides good stability and also allows subsequent sterilization, both of which are key from a standpoint of automation, standardization, and clinical implementation.

The stabilizing agent may be located on any surface of the collection device. The stabilizing agent may also be located on stoppers, seals for closing such devices or on mechanical, component surfaces and sub-surfaces, or other, inserts placed within such devices. Preferably, the stabilizing agent is located anywhere along at least one interior wall of the collection device or anywhere within the reservoir portion. In addition, some phosphatase inhibitors may exhibit light sensitivity. Thus, it may be desirable to protect the agent from light. For such inhibitors, use of an opaque tube, e.g., an amber-colored tube with or without observation window, would be advantageous (Kirk, Scott, consider window on amber tube for independent claim). Alternatively, placing the agent into a capsule that protects it from light exposure, e.g., in powdered form, and then placing the capsule into the tube would also address this issue. Capsulating the agent may also prevent other undesirable interactions between the agent and other elements in the container. Capsule materials that dissolve upon sample collection are well known in the art.

The stabilizing agent may be applied to the collection device by any number of methods. For example, the stabilizing agent may be spray dried, loosely dispensed or lyophilized over the surface of the interior wall of the collection device. Alternatively, the stabilizing agent, such as when in gel or liquid form, for example, may be positioned in the reservoir portion of the collection device. Additional methods for providing the collection device with the stabilizing agent are also possible. Typically, to dispose the desired amount of agent into a container, one reconstitutes a solid form of the agent and then dispenses the appropriate amount of liquid into the container. The liquid may be spray dried, disposed into the bottom of the container or subsequently lyophilized.

The quantity and location of the stabilizing agent are determined by several variables, including the mode of application, the specific stabilizing agent used, the internal volume and internal pressure of the collection device, and the volume of the biological sample drawn into the container.

The concentration of the stabilizing agent is sufficient to stabilize the protein and to inhibit or prevent protein degradation.

In addition to the stabilizing agent, the device of the present invention may also contain carrier media (e.g., water or alcohol), stabilizing media (e.g., polyvinylpyrollidone, trehalose mannitol, etc.) and/or one or more other additives for treating the biological sample. Suitable additives include, but are not limited to, phenyl, phenyl/chloroform mixtures, alcohols, aldehydes, ketones, organic acids, salts of organic acids, alkali metal salts of halides, organic chelating agents, fluorescent dyes, antibodies, binding agents, anticoagulants such as sodium citrate, heparin, potassium EDTA and the like, and any other reagent or combination of reagents normally used to treat biological samples for analysis. Other potential additives include antioxidants and reducing agents, which may help preserve protein confirmation, e.g., preserve sulfhydryl group couplings. It may also be advantageous to include a buffering agent or sugar compounds. Yet other additive groups or chemistries include those that enhance solubility of the preservative or stabilizer additive in the specimen matrix. Preferably, the carrier and additives do not degrade proteins. Where the stabilizing agent is in tablet form, pharmaceutical tablet disintegrating materials, which are known to those skilled in the art, may be included, if desired.

The methods of the present invention include obtaining a biological sample and introducing the sample into the container containing the stabilizing agent. In preferred embodiments, the biological sample is withdrawn from the patient directly into the collection container without any intervening process steps. It has been found that collecting the biological sample directly from the patient, such as when collecting a whole blood sample, and introducing the sample directly into the container containing the stabilizing agent substantially reduces or prevents the modification, degradation and/or fragmentation of proteins that otherwise occurs when the sample is stored before combining it with the stabilizing agent. The method of the present invention is useful both with open collection systems and with closed collection systems wherein the opening is closed by a closure means.

In a preferred embodiment, the collection device of the present invention is for drawing a whole blood sample directly from a patient for stabilizing the proteins immediately at the point of collection. The device may be an evacuated system for collecting blood. Alternatively, the device may be a partially-evacuated or a non-evacuated system for collecting blood. A suitable example of an evacuated system is a closed tube. A manual syringe draw is a suitable example of both a partially-evacuated and a non-evacuated system. Non-evacuated systems may also include automatic draw systems. Evacuated systems are particularly preferred.

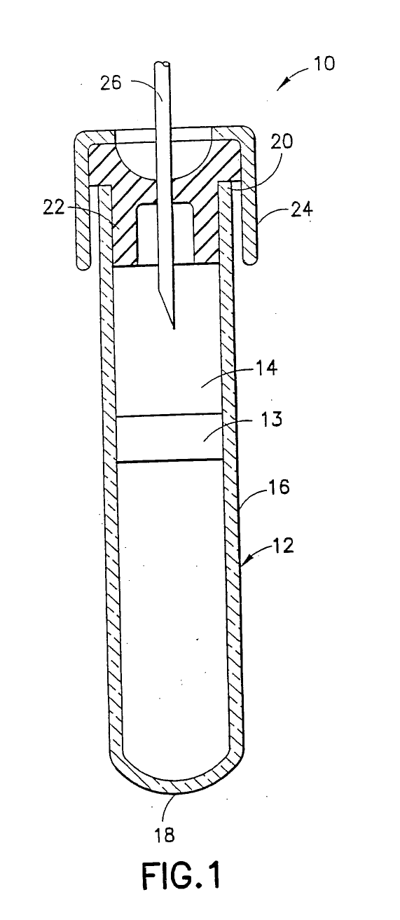

Referring to the drawings in which like reference characters refer to like parts throughout the several views thereof, FIG. 1 shows a typical blood collection device 10, which includes a container 12 defining an internal chamber 14. In the embodiment illustrated, container 12 is a hollow tube having a side wall 16, a closed bottom end 18 and an open top end 20. Optionally, a separating member 13 is provided within the container chamber 14. Separating member 13 serves to assist in separating components of the sample, for example, by centrifugation. Container 12 is dimensioned for collecting a suitable volume of biological fluid, preferably blood. A closure means 22 for covering open end 20 to close container 12 is necessary where a sterile product is demanded. For conventional tubes, a screw cap is normally sufficient. For evacuated collection tubes, a tight-fitting, elastomeric plug is generally employed to contain the vacuum during the required storage periods. Preferably, closure 22 forms a seal capable of effectively closing container 12 and retaining a biological sample in chamber 14. Closure 22 may be one of a variety of forms including, but not limited to, rubber closures, metallic seals, metal-banded rubber seals and seals of different polymers and designs. A protective shield 24 may overlie closure 22. Container 12 also contains a stabilizing agent in accordance with the present invention.

Container 12 can be made of glass, plastic or other suitable materials. Preferably, container 12 is transparent. Non-limiting examples of suitable transparent thermoplastic materials for container 12 are polycarbonates, polyethylene, polypropylene and polyethyleneterephthalate. Plastic materials can be oxygen impermeable materials or may contain an oxygen impermeable or semi-permeable layer. Alternatively, container 12 can be made of a water and air permeable plastic material. The stabilizing agent may be provided to the container using any appropriate means. In one aspect, the stabilizing agent is in a liquid solution and is placed into the container. Subsequently, the solution may be lyophilized by methods that are known in the art such as, for example, freeze drying. For example, by freezing the solution and then slowly warming after freezing, while simultaneously applying a vacuum, a freeze-dried powder remains in the collection tube. An additive such as an excipient, for example, PVP or trehalose, may also be added to the stabilizing agent solution prior to freeze drying so that the resulting stabilizing agent is pelletized in the container. Vacuum drying may also be used after adding the stabilizing solution. In another aspect, the stabilizing agent is formed into a liquid or solid aerosol and sprayed onto one or more surfaces of the interior of the container.

The pressure in chamber 14 is selected to draw a predetermined volume of biological sample into chamber 14. Preferably, closure 22 is made of a resilient material that is capable of maintaining the internal pressure differential between atmospheric pressure and a pressure less than atmospheric. Closure 22 is such that it can be pierced by a needle 26 or other cannula to introduce a biological sample into container 12 as known in the art. Preferably, closure 22 is resealable. Suitable materials for closure 22 include, for example, silicone rubber, natural rubber, styrene butadiene rubber, ethylene-propylene copolymers and polychloroprene.

Suitable examples of container 12 include single-wall and multi-layer tubes. A more specific example of a suitable container 12 is disclosed in U.S. Pat. No. 5,860,937 to Cohen, which is hereby incorporated by reference in its entirety.

A useful manufacturing process for devices according to the present invention involves obtaining a collection container; adding at least one phosphatase inhibitor to the container; lyophilizing the at least one inhibitor; evacuating the container; and sterilizing the container. The at least one inhibitor may be dispensed into the container in solution form. After adding the inhibitor to the collection container, a separating member may be added to the container, if desired.

As noted, container 12 may also contain a gel, mechanical or other separating member (e.g., a filter mechanism). In such cases, the stabilizing agent may be spray dried and/or lyophilized on an exterior surface of the separation media Container 12 may also be a collection device for blood plasma preparation. Such a collection device comprises, in addition to the stabilizing agent, an element for separating plasma from human or animal whole blood. The element for separating plasma from whole blood may be a separating member such as a gel formulation, a mechanical media or a filter mechanism. The gel is desirably a thixotropic polymeric gel formulation. The gel may be a homopolymer or a copolymer and may include silicone-based gels such as, for example, polysiloxanes, or organic hydrocarbon-based gels such as, for example, polyacrylics, polyesters, polyolefins, oxidized cis polybutadienes, polybutenes, blends of epoxidized soybean oil and chlorinated hydrocarbons, copolymers of diacids and propandiols, hydrogenated cyclopentadienes and copolymers of alpha-olefins with dialkylmaleates. The gel desirably isolates the plasma from the cells of the blood sample in the tube by serving as a density separation medium. An example of a suitable plasma preparation tube is disclosed in U.S. Pat. No. 5,906,744 to Carroll et al., which is hereby incorporated by reference in its entirety. In this way, stabilization can be provided both before, during and after centrifugation to separate the plasma from the blood. In the case of a gel separating material, it may be desirable to provide physical/chemical separation between the stabilizing agent and the gel, e.g., use of a capsule as discussed above. For example, if portions of the agent are incorporated into or react with the gel, the effectiveness of the agent may be reduced. For the same reasons, where a mechanical separating element is used, the element is desirably substantially inert to the stabilizing agent, and this reflects a significant advantage of such a separator. Providing a separating element in plasma tubes, versus centrifuging without a separating element, is particularly advantageous. Specifically, because cell lysing releases proteases that degrade proteins of interest, the better the separation between the cells (i.e., the clotted blood) and the plasma, the better the stability of proteins in the plasma sample. Useful mechanical separators are found, for example, in U.S. Pat. Nos. 6,516,953; 6,406,671; 6,409,528; and 6,497,325, the contents of which are hereby incorporated by reference in their entirety. Useful filter mechanisms are found, for example in U.S. Pat. No. 6,506,167 the contents of which are hereby incorporated by reference in their entirety. Ishimito et al. disclose a blood separating tube including an upstream tube separated by a filter from a downstream tube where the tubes are attachable to and detachable from each other and are evacuated. During blood collection, blood is removed from a patient through intravenous puncture and transferred into the upstream tube through blood pressure and negative pressure inside the tube. In accord with the disclosure, a pressure differential is supposed to be created between the upstream tube and the downstream tube as the blood contacts the filter between the two tubes. Thus centrifugation is not required in order to separate the whole blood. Several suggested filters include a membrane, glass fibers, filter paper with large pores having attached thereto anti-hemocyte antibodies, a filter impregnated with a cationic macromolecular substance to aggregate cells, and a laminated multi-layer filter.

Container 12 may also be a collection tube for centrifugally separating lymphocytes and monocytes from heavier phases of a sample of whole blood comprising, in addition to the stabilizing agent, a liquid density gradient medium and a means for preventing mixing of the liquid density gradient medium with a blood sample prior to centrifugation. An example of a suitable lymphocyte/monocyte collection tube is disclosed in U.S. Pat. No. 5,053,134 to Luderer et al., which is hereby incorporated by reference in its entirety.

In another embodiment, the invention provides a kit having at least two containers comprising one or more stabilizing agents. For example, the kit may comprise a primary collection tube, e.g., a plasma separating tube having a separating element therein, and a secondary tube for testing, e.g., for pouring or otherwise dispensing the collected plasma into. Both would have stabilizing agent(s) therein, to ensure that the proteins of interest remained stable throughout. The tube may have stages whereby certain proteins are separated or depleted in one stage and stabilized in another. Optionally, the kit could include a tube-to-tube transfer device to prevent the need for pouring or other unsafe transfer practices, in which case the secondary tube would be at a reduced pressure to draw in the plasma. One using such a kit would collect a sample in the primary tube, centrifuge, transfer the sample of interest to the secondary testing tube, and perform the testing. The secondary testing tube could be of a variety of sizes, depending on the desired testing.

In another embodiment, the container is a tube with two open ends having closures thereon. Such a tube would allow one to sample, e.g., for a plasma separating tube with a separating element therein, either the plasma sample or the clot sample.

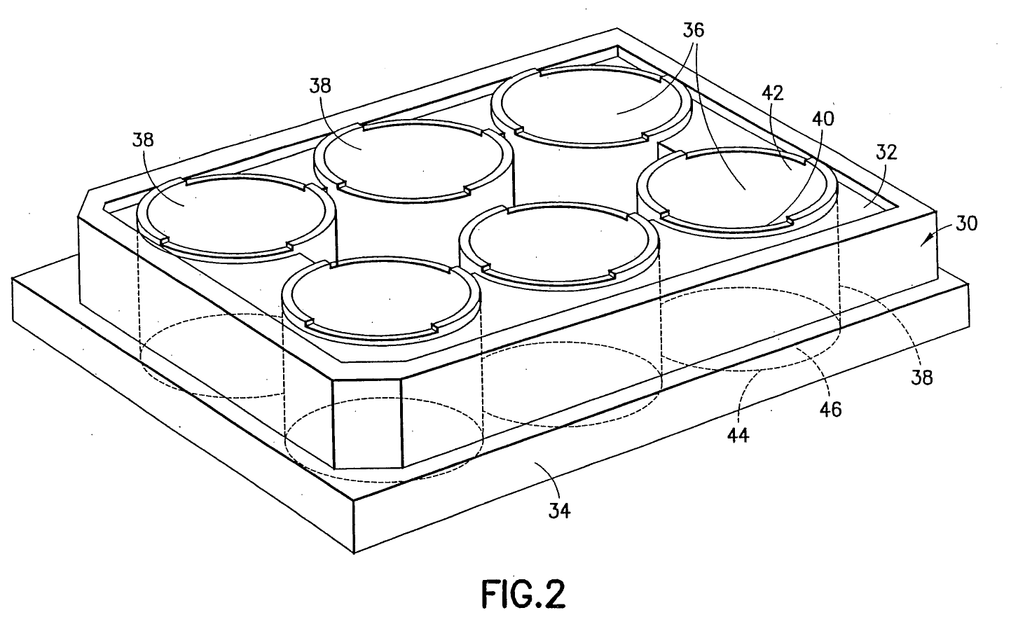

In yet another embodiment, the collection device of the present invention comprises a test plate such as, for example, a single- or multi-well plate, a microtiter plate, a tissue culture plate or the like. A typical test plate generally comprises one or more wells, which are preferably cylindrical. As shown in FIG. 2, a test plate 30 includes an upper surface 32 and a lower surface 34. Test plate 30 further includes a number of wells 36 each comprising a sidewall 38 extending from upper surface 32 of the plate to lower surface 34 of the plate. Each well comprises a top portion 40 and a bottom portion 44. Top portion 40 comprises an open end 42 that extends to bottom portion 44, which comprises a closed end 46. Bottom portion 44 may be flat, conical (pointed) or rounded. The capacity of each well 36 typically ranges from several milliliters (ml) to less than about 0.5 ml. Wells 36 may each accommodate therein a stabilizing agent according to the present invention.

The number of wells 36 in test plate 30 is not critical. There may be any number of wells, although six-, twelve-, twenty-four-, forty-eight- and ninety-six-well test plates are commonly known and available. In FIG. 2, a six-well test plate is illustrated, merely for exemplary purposes, and the invention is not dependent upon the number of wells. Most standard multi-well plates have the wells arranged in orthogonal rows and columns so as to be able to clearly identify the individual wells being used. Of course, the arrangement of the wells in test plate 30 is not an essential limitation of the present invention because any arrangement of wells is contemplated by the invention.

Plate 30 may be formed from thermoplastic materials by vacuum forming, sheet molding, injection molding or other similar techniques. Suitable thermophstic materials include, but are not limited to, polystyrene, polyvinylchloride, polycarbonate, polyethyleneterephthalate and the like. Preferably, plate 30 is transparent.

Surrounding the wells and forming the outside border of test plate 30 are sidewalls 38. In the present embodiment, test plate 30 has six (6) sidewalls. Well known test plates are rectangle or quadrilaterally shaped, although for purposes of the present invention the plate may be fabricated in any practical configuration. Examples of suitable test plates containing a plurality of wells are disclosed in U.S. Pat. No. 5,882,922 to Tyndorf et al., U.S. Pat. No. 5,801,055 to Henderson and U.S. Pat. No. 5,681,743 to Brian et al., each of which is hereby incorporated by reference in its entirety.

In yet another embodiment, the collection device according to the present invention may be a sample collection assembly for the collection, transport and dispensing of biological samples. The collection assembly generally includes a plurality of sample wells for collecting individual biological samples. The sample wells are supported in a sample tray in a spaced-apart orientation. The sample tray may be supported within a case that encloses the sample tray and allows the safe and efficient transport of the sample wells. The sample tray is movably accommodated within the case for movement between a first position enclosing the plurality of sample wells, to a second position rendering exteriorly accessible one of the sample wells so that the sample can be manually dispensed from the tray.

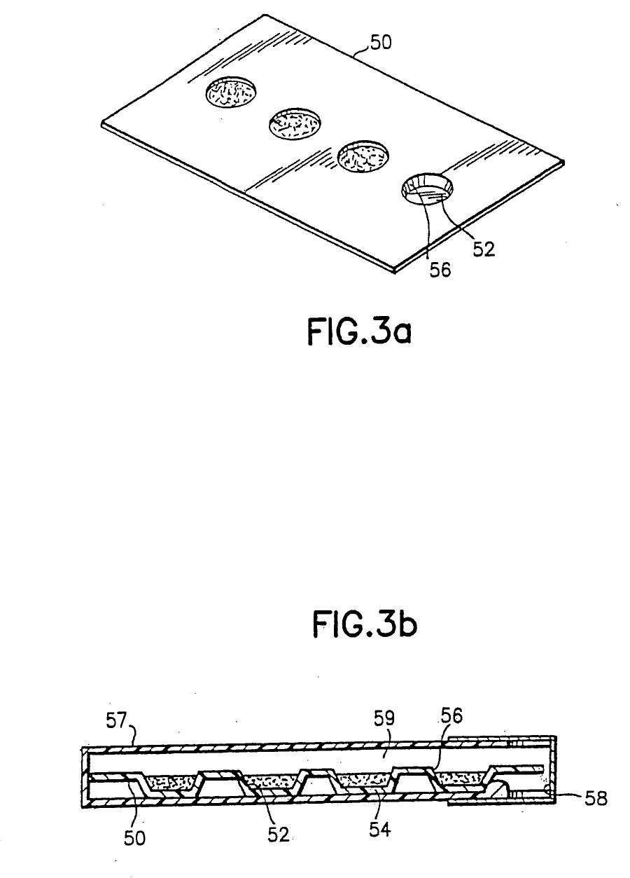

As shown in FIGS. 3a and 3b, sample tray 50 includes a plurality of longitudinally spaced depressions forming specimen collection wells 52. Sample tray 50 may be formed of a suitably deformable plastic material. Wells 52 have a bottom 54 and an open end 56. It is contemplated that the sample wells may be in the shape of open ended cup-like members. Wells 52 are constructed to have sufficient depth so as to retain a suitable volume of a biological sample. Wells 52 may each accommodate therein a stabilizing agent according to the present invention. While tray 50 of the present invention is shown having a single row of wells 52 formed therein, the present invention contemplates that the wells may be provided in any number or any array desirable for a particular testing situation. The sample collection assembly may include a sample collection case 57. Upon collection of a biological sample within wells 52, sample tray 50 may be inserted into the open end 58 of sample collection case 57 and then within the interior 59 of sample collection case 57 until all of wells 52 are enclosed therein. A suitable sample collection assembly is disclosed in U.S. Pat. No. 6,357,583 B1 to Rainen, which is hereby incorporated by reference in its entirety.

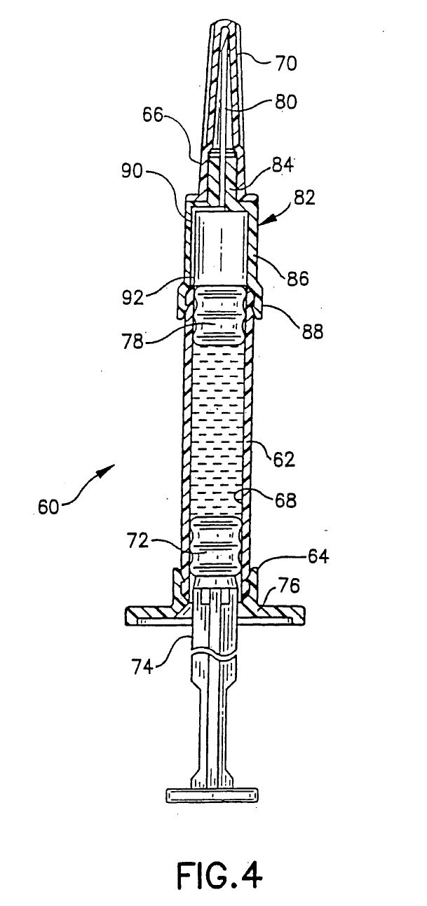

According to another embodiment of the present invention, as depicted in FIG. 4, the collection device comprises a syringe and, more preferably, a syringe pre-filled with a stabilizing agent in accordance with the present invention. A typical syringe comprises a generally cylindrical barrel having opposed proximal and distal ends with at least one chamber formed between the ends for receiving a substance such as a biological sample. A plunger is typically sealably disposed within the barrel and movable with respect thereto, and sealing means may be sealably disposed approximate to the distal end of the barrel. Referring now to FIG. 4, there is shown a syringe 60, which includes an elongate barrel or cylinder 62 having an open, proximal end 64 and a distal end 66, with at least one hollow chamber 68 formed between the proximal and distal ends for receiving a biological sample. In the embodiment illustrated, distal end 66 includes a needle guard 70. The needle guard keeps the syringe, as well as the needle, sterile during storage.

The barrel of the syringe includes a stabilizing agent. Preferably, the barrel of the syringe is pre-filled with the stabilizing agent. Pre-filled syringes, as the term is known in the art, are syringes that are filled by the manufacturer and shipped to the health care provider ready for use.

A plunger 72 may be situated at open, proximal end 64. Plunger 72 can be moved by means of a plunger rod 74, which is secured to the plunger, for example, by screwing. At the same end where the plunger is situated, the barrel may have a fingergrip 76, which is secured to the barrel according to the so-called snap-cap principle. Fingergrip 76 preferably consists of slightly resilient material, for example plastics. In another embodiment (not shown), the fingergrip is a flange-like part of the barrel projecting radially outwards. Of course, other constructions known to those skilled in the art are possible.

A stopper 78, which closes the barrel, may be situated in the end of the barrel remote from the plunger. The plunger and the stopper are preferably manufactured from an elastic material and, most preferably, from rubber of a pharmaceutical quality.

In the embodiment illustrated, an injection needle 80 is secured to the barrel by means of a needle holder 82. The needle holder has a neck 84, which holds the needle, a shaft 86 and a collar 88. The needle holder is preferably manufactured from slightly resilient material that has resistance to deformation such as, for example, plastics, and is secured to the end of the barrel by means of a snap-cap construction. In the alternative, the needle holder may be secured to the barrel by means of a screwed or adhesive connection or, when the barrel also comprises a collar, by means of a clamping ring. In the latter embodiment, the needle holder may also be flanged around a collar of the barrel.

Although the syringe barrel illustrated in this embodiment includes a locking Luer-type collar 88, it is within the purview of the present invention to include syringe barrels without a collar, syringe barrels having an eccentrically positioned nozzle and various other nozzle-like structures adapted to accept, either permanently or removably, a needle cannula or needle cannula assembly. It is only required that there is an aperture on the distal end of the syringe barrel in fluid communication with the interior of the syringe barrel.

One or more slots 90 may be recessed in the inner wall of shaft 86 and the rear face of neck 84. The slot or slots extend into the rear end of the cannula. In cross-section, the slots may be parts of a circle, but other shapes are also possible, provided the size is such that sufficient injection liquid can be readily passed through; this is achieved if the diameter of ihe slot or the overall cross-section of the slots is at least as large as that of the cannula. Shaft 86 of needle holder 82 is constructed so that when stopper 78 slides axially forward, it is received, with friction, by the shaft; therefore, apart from slots 90 recessed in the shaft, the inside diameter of the shaft is approximately as large as that of barrel 62. Shaft 86 of needle holder 82 is slightly longer than stopper 78 so that the part 92 of the slot(s) adjoining the barrel is free when the stopper is moved forward against the rear wall of the neck of the needle holder. If desired, needle guard 70 may be constructed to also serve as a plunger rod. In that case, prior to use of the syringe, the needle guard is removed from the needle and secured at the other end of the syringe to the plunger.

Generally, a syringe comprising a needle protector has a safety member, which indicates whether the needle protector has previously been removed. Such a safety member in the form of a cap is described in, for example, U.S. Pat. No. 3,995,630.

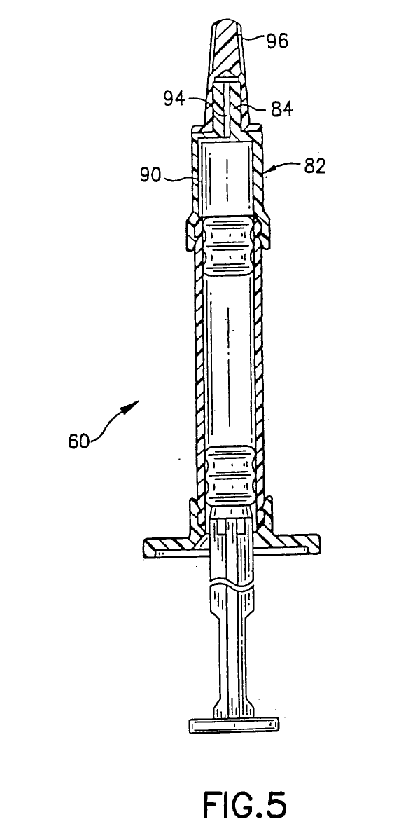

In further embodiments, the syringe is not stored with a needle in position, i.e., it is a needleless syringe as known in the art. This is illustrated in FIG. 5. With such a syringe, before use, the needle is positioned on neck 84 of needle holder 82 by means of a needle hub. A so-called Luer cone is preferably used for this connection. In this embodiment, aperture 94 in the neck of the needle holder is closed on the outside by a protective cap 96, which ensures the sterility of the syringe as well as the needle holder. Slot 90 recessed in the needle holder projects into the end of the neck aperture.

An example of a suitable syringe is disclosed in U.S. Pat. No. 6,027,481 to Barrelle et al., which is hereby incorporated by reference in its entirety. Other examples of suitable syringes are disclosed in, for example, U.S. Pat. No. 4,964,866 to Szwarc, U.S. Pat. No. 4,986,818 to Imbert et al., U.S. Pat. No. 5,607,400 to Thibault et al. and U.S. Pat. No. 6,263,641 B1 to Odell et al., each of which is hereby incorporated by reference in its entirety.

In a further embodiment, the collection device of the present invention comprises a catheter. As known in the art, catheters are commonly employed when a patient requires repeated doses of medication or other substances. A catheter permits repeated and continuous administration of medication directly into a patient's blood stream, or other region of the body, without repeated injections. Typically, catheters have a hollow tubular lumen, a proximal end and a distal end. The distal end of the catheter, which may be open or closed, is inserted into the vein or artery of a patient.

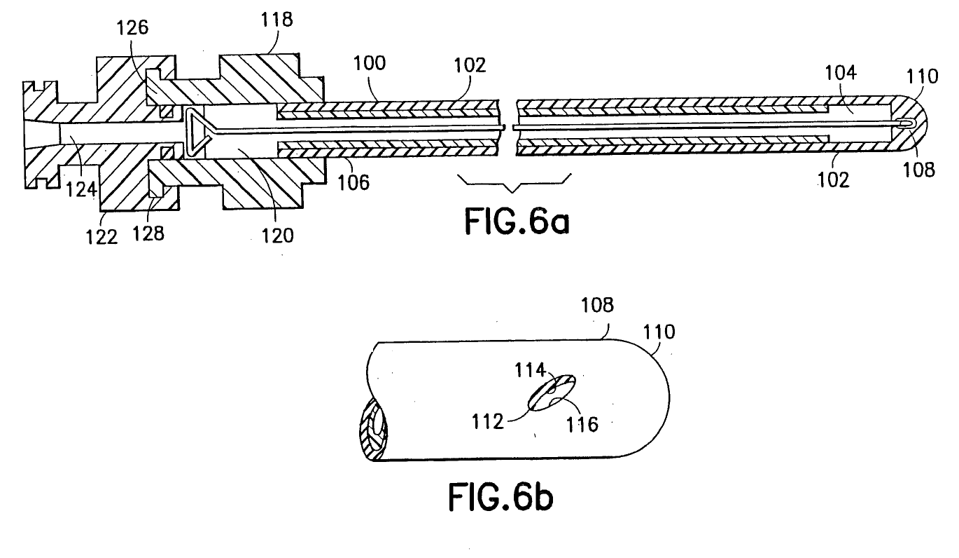

FIG. 6a illustrates an exemplary catheter assembly that includes a flexible catheter 100 having a cylindrical side wall 102 describing a lumen 104 therethrough, a proximal end 106 and a closed distal end 108 which, in this illustrated embodiment, has a rounded exterior surface 110 to facilitate insertion of the catheter into the patient. As illustrated in FIG. 6b, catheter 100 includes a slit 112 through side wall 102 adjacent to distal end 108 and is defined by two opposed faces 114 and 116 formed in the side wall. Catheter 100 includes a stabilizing agent according to the present invention, preferably in the lumen of the catheter.

The proximal end of the catheter is connected to a catheter housing 118 having a conduit 120 therethrough. Conduit 120 in the catheter housing and lumen 104 in the catheter are in fluid communication. A valve control knob 122 having a passageway 124 therethrough is rotatably connected to catheter housing 118 so that passageway 124 is in fluid communication with conduit 120. Valve control knob 122 and catheter housing 118 are held together by virtue of proximal flange 126 on the catheter housing which, engages rotational groove 128 in the valve control knob. This structure allows the valve control knob to rotate with respect to the catheter housing but keeps the two elements from coming apart. An example of a suitable catheter is disclosed in U.S. Pat. No. 4,737,152 to Alchas, which is hereby incorporated by reference in its entirety.

In yet a further embodiment, the collection device of the present invention comprises a pipette. In laboratory settings, it is well known to use a pipette to extract a certain volume of a biological fluid from one container and to transport and dispense some or all of the extracted volume into another container. Typically, pipettes are generally hollow tubular members that are used by applying suction at an open upper end, or mouthpiece, in order to extract or aspirate a quantity of fluid medium into the hollow tube. A pressure differential maintained by closing the mouthpiece opening retains the fluid within the pipette allowing transport of the fluid medium to another container. Selective opening of the mouthpiece allows a quantity of the fluid medium contained in the pipette to be dispensed A certain degree of accuracy in the amount of fluid dispensed is provided by the tapered end portions by reducing the amount of fluid lost due to dripping.

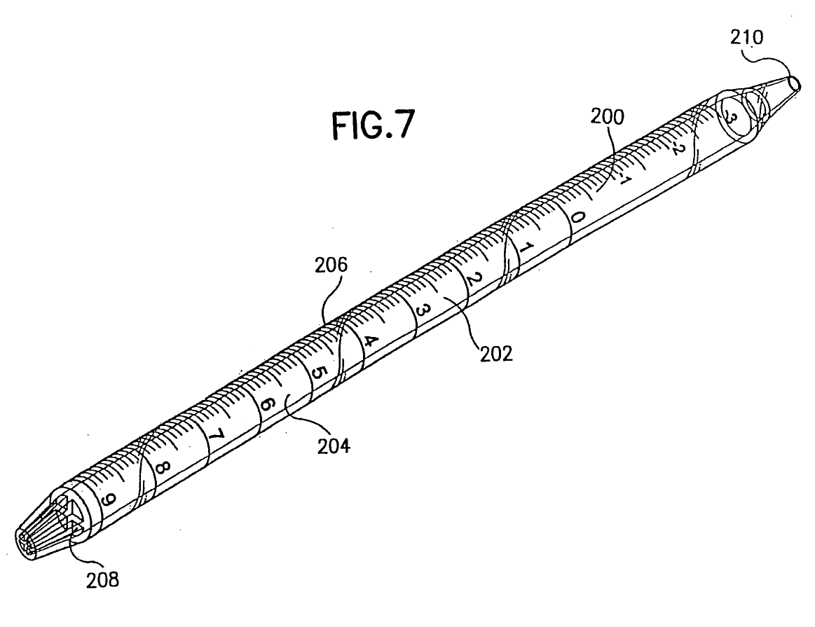

Referring now to FIG. 7, an exemplary pipette 200 is shown. Pipette 200 is generally an elongate tubular member defined by a tubular wall 202 of generally uniform thickness. Within tubular wall 202, a pipette interior 204 is defined for accommodating a given volume of fluid medium, for example, a biological sample. Pipette 200 includes an elongate generally cylindrical main body portion 206 that is coextensive with interior 204. Pipette body 206 may be pre-filled with a stabilizing agent according to the present invention.

In order to aspirate and dispense a biological fluid, pipette 200 includes a dispensing portion 208 at one end of body 206 and a mouthpiece 210 at the other end. Both dispensing portion 208 and mouthpiece 210 are in communication with interior 204 of pipette 200 so as to permit aspirating and dispersing of the fluid through dispensing portion 208 by creating a selective pressure differential within interior 204 of pipette 200 using mouthpiece 210. Such a pressure differential can be created manually by opening and closing mouthpiece 210 or may be created by use of mechanical pipette aids.

Pipette 200 may be constructed of glass or a thermoplastic material such as polycarbonate, polyethylene, polyester, polystyrene, polypropylene, polysulfone, polyurethane, ethylene vinyl acetate or the like. Thermoplastic pipettes have largely replaced glass pipettes for many uses. The material of pipette 200 may be transparent, translucent or opaque.

Examples of suitable pipettes are disclosed in, for example, U.S. Pat. No. 6,280,689 B1 to Stevens and U.S. Pat. No. 6,343,717 B1 to Zhang et al., both of which are hereby incorporated by reference in their entirety.

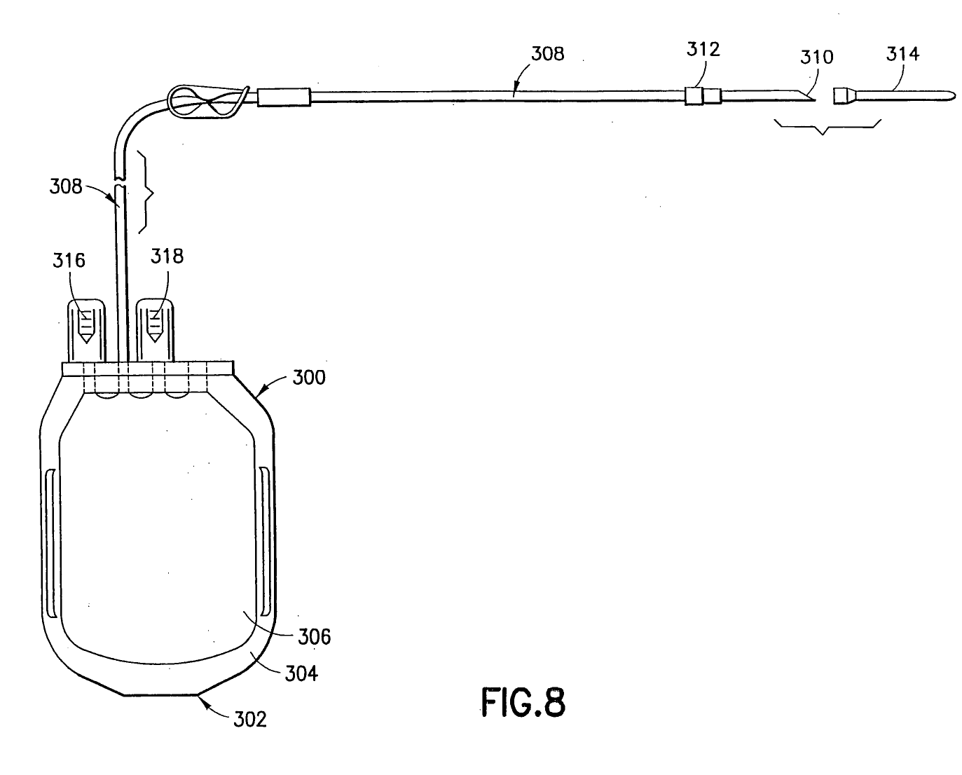

The collection device of the present invention may also comprise a collection bag suitable for holding a biological sample such as, for example, a blood collecting bag, a blood plasma bag, a buffy coat bag, a platelet bag or the like. For ease of description, a blood collecting bag will now be described with reference to FIG. 8.

FIG. 8 illustrates a blood collecting bag 300 for accommodating collected blood. Blood collecting bag 300 has a body 302 formed by superposing a pair of identically cut pieces of a sheet material made of a resin, which will be more specifically described hereinafter, and possessed of flexibility and fusing (i.e., heat fusion, high frequency fusion or the like) or adhesively joining to each other the periphery of the sealing portion 304 of each of the pieces of sheet material. A blood-accommodating portion 306 accommodating collected blood is formed at an inner portion surrounded with sealing portion 304 of body 302. Blood collecting bag 300 preferably contains a stabilizing agent in accordance with the present invention.

One end of the flexible tube 308 communicating with blood-accommodating portion 306 is connected with body 302 at an upper portion thereof. A blood collecting needle 310 is installed at the other end of flexible tube 308 through a hub 312. A cap 314, which is to cover blood collecting needle 310, may be installed on hub 312. Two openings 316 and 318, each sealed with a peel tab, may be formed at an upper portion of body 302 such that they can be opened.

The composition, characteristics and the like of the material of the sheets composing body 302 of blood collecting bag 300 are not limited to specified ones. In this case, as the sheet material composing blood collecting bag 300, soft polyvinyl chloride or materials containing the soft polyvinyl chloride as their main component is preferably used. For example, a copolymer containing the soft polyvinyl chloride as its main component and a small amount of macromolecular material, a polymer blend, a polymer alloy and the like can be used. As the plasticizer for the soft polyvinyl chloride, dioctylphthalate (DEHP, di(2-ethylhexyl)phthalate) and (DnDP, di(n-decyl)phthalate) can be preferably used. The content of such a plasticizer in the polyvinyl chloride is preferable to be in the approximate range of 30 to 70 parts by weight, based on 100 parts by weight of polyvinyl chloride.

The other substances that are effectively usable for the sheet material of blood collection bag 300 are polyolefins, i.e., the products of homopolymerization or copolymerization of such olefins or diolefins as ethylene, propylene, butadiene and isoprene. Typical examples include polyethylene, polypropylene, ethylene vinyl acetate copolymer (EVA), polymer blends formed between EVA and various thermoplastic elastomers and arbitrary combinations thereof. Such polyesters as polyethylene terephthalate (PET), polybutylene terephthalate (PBT), poly-1,4-cyclohexane dimethyl terephthalate (PCHT) and polyvinylidene chloride are also usable.

In yet another embodiment, the collection device of the present invention may be a laboratory vessel that contains the stabilizing agent. Particular vessels that can be used in accordance with the present invention include, for example, vials, flasks, spinner flasks, roller bottles, microscope slides, microscope slide assemblies, sample chambers for analytical devices, tapes, laminates, arrays, tubing and the like. Laboratory vessels according to the present invention have at least one operational surface. Many vessels according to the invention have at least one interior wall, which defines a reservoir portion for containing the biological sample, and at least one opening in communication with the reservoir portion.

Plastic or glass is often used to manufacture the laboratory vessels. Some preferred materials used to manufacture laboratory vessels include polypropylene, polyethylene, polyethyleneterephthalate, polystyrene, polycarbonate and cellulosics. Because polypropylene is inexpensive, it is a particularly preferred material for laboratory vessels used for handling and transporting minute and precise amounts of biological sample.

Examples of other suitable materials for the laboratory vessels of the present invention include polyolefins, polyamides, polyesters, silicones, polyurethanes, epoxies, acrylics, polyacrylates, polyesters, polysulfones, polymethacrylates, PEEK, polyimide and fluoropolymers. Glass products including silica glass are also used to manufacture laboratory vessels.

While various embodiments have been chosen to demonstrate the invention, it will be understood by those skilled in the art that various modifications and additions can be made without departing from the scope of the invention as defined in the appended claims.

1. An apparatus for collecting a biological sample, comprising:
a container having a reservoir portion for receiving the sample; and
at least one protein stabilizing agent comprising at least one phosphatase inhibitor in the reservoir of the container.

2. The apparatus of claim 1, wherein the container is selected from the group consisting of tubes, closed system blood collection devices, collection bags, syringes, pre-filled syringes, catheters, microtiter plates, multi-well collection devices, flasks, spinner flasks, roller bottles, vials, pipettes, pipette tips and tissue and other biological sample collection containers.

3. The apparatus of claim 1, wherein the container is a tube having a first end and a second end.

4. The apparatus of claim 3, further comprising a separating member disposed in the container.

5. The apparatus of claim 4, wherein the separating member is a mechanical separating element.

6. The apparatus of claim 5, wherein the mechanical separating element is at least partially coated with the at least one stabilizing agent.

7. The apparatus of claim 5, wherein the mechanical separating element is substantially inert with respect to the stabilizing agent.

8. The apparatus of claim 4, wherein the separating member is a gel.

9. The apparatus of claim 8, wherein the gel separating member is physically separated from the stabilizing agent.

10. The apparatus of claim 1, wherein the protein stabilizing agent is in a form selected from the group consisting of a solution, suspension or other liquid, a pellet, a tablet, a capsule, a spray-dried material, a freeze-dried material, a powder, a particle, a gel, crystals or a lyophilized material.

11. The apparatus of claim 10, wherein the protein stabilizing agent is lyophilized.

12. The apparatus of claim 1, wherein the at least one phosphatase inhibitor, inhibits at least one phosphatase selected from the group consisting of serine phosphatase, threonine phosphatase, and tyrosine phosphatase.

13. The apparatus of claim 1, wherein the stabilizing agent further comprises at least one protease inhibitor.

14. The apparatus of claim 1, wherein the stabilizing agent comprises a phosphatase inhibitor cocktail.

15. The apparatus of claim 1, wherein the stabilizing agent comprises more than two phosphatase inhibitors.

16. The apparatus of claim 1, further comprising a carrier media.

17. The apparatus of claim 1, further comprising a stabilizing media.

18. The apparatus of claim 17, wherein the stabilizing media is trehalose.

19. The apparatus of claim 1, further comprising at least one antioxidant.

20. The apparatus of claim 1, further comprising at least one reducing agent.

21. The apparatus of claim 1, further comprising at least one buffering agent.

22. The apparatus of claim 3, wherein the first end is an open end, the second end is a closed end, and further comprising a closure means for sealing the first open end.

23. The apparatus of claim 3, wherein the first end is an open end, the second end is an open end, and further comprising a first closure means for sealing the first open end and a second closure means for sealing the second open end.

24. The apparatus of claim 3, wherein the protein stabilizing agent is lyophilized.

25. The apparatus of claim 24, wherein the protein stabilizing agent comprises more than two phosphatase inhibitors.

26. The apparatus of claim 24, wherein the tube further comprises an anticoagulant.

27. The apparatus of claim 26, wherein the anticoagulant is spray-dried onto at least a portion of an interior wall.

28. The apparatus of claim 26, wherein the anticoagulant comprises a salt of EDTA.

29. The apparatus of claim 26, wherein the anticoagulant comprises heparin.

30. A tube for collecting and stabilizing a biological sample, comprising:
a first end, a second end and at least one interior wall defining a reservoir portion for receiving the sample;
at least one protein stabilizing agent in the reservoir of the container, the agent comprising at least one phosphatase inhibitor;
a thixotropic polymeric gel in the reservoir; and
an element for maintaining separation of the protein stabilizing agent and the gel.

31. The tube of claim 30, wherein the element for maintaining separation is a capsule.

32. The tube of claim 31, wherein the first end is an open end and the second end is a closed end.

33. The tube of claim 32, further comprising a closure means for sealing the first open end.

34. The tube of claim 33, wherein the closure means is pierceable by a needle for supplying the sample to the tube.

35. The tube of claim 33, wherein the tube is partially evacuated.

36. The tube of claim 35, wherein the protein stabilizing agent is lyophilized.

37. The tube of claim 36, wherein the protein stabilizing agent comprises more than two phosphatase inhibitors.

38. The tube of claim 36, wherein the tube further comprises an anticoagulant spray-dried onto at least a portion of the interior wall.

39. The tube of claim 30, wherein the agent further comprises at least one protease inhibitor.

40. The tube of claim 31, wherein the first end is an open end and the second end is an open end.

41. The tube of claim 40, further comprising a first closure means for sealing the first open end and a second closure means for sealing the second open end.

42. The tube of claim 41, wherein both closure means are pierceable by a needle for supplying the sample to the tube.

43. The tube of claim 41, wherein the tube is partially evacuated.

44. The tube of claim 43, wherein the protein stabilizing agent is lyophilized.

45. The tube of claim 44, wherein the protein stabilizing agent comprises more than two phosphatase inhibitors.

46. The tube of claim 44, wherein the tube further comprises an anticoagulant spray-dried onto at least a portion of the interior wall.

47. A tube for collecting and stabilizing a biological sample, comprising:
a first end, a second end and at least one interior wall defining a reservoir portion for receiving the sample;
at least one protein stabilizing agent in the reservoir of the tube, the agent comprising at least one phosphatase inhibitor; and
a mechanical separating element in the reservoir.

48. The tube of claim 47, wherein the mechanical separating element is substantially inert with respect to the stabilizing agent.

49. The tube of claim 47, wherein the first end is an open end and the second end is a closed end.

50. The tube of claim 49, further comprising a closure means for sealing the first open end.

51. The tube of claim 50, wherein the closure means is pierceable by a needle for supplying the sample to the tube.

52. The tube of claim 50, wherein the tube is partially evacuated.

53. The tube of claim 52, wherein the protein stabilizing agent is lyophilized.

54. The tube of claim 53, wherein the protein stabilizing agent comprises more than two phosphatase inhibitors.

55. The tube of claim 53, wherein the tube further comprises an anticoagulant spray-dried onto at least a portion of the interior wall.

56. The tube of claim 55, wherein the first end is an open end and the second end is an open end.

57. The tube of claim 56, further comprising a first closure means for sealing the first open end and a second closure means for sealing the second open end.

58. The tube of claim 57, wherein both closure means are pierceable by a needle for supplying the sample to the tube.

59. The tube of claim 57, wherein the tube is partially evacuated.

60. The tube of claim 59, wherein the protein stabilizing agent is lyophilized.

61. The tube of claim 60, wherein the protein stabilizing agent comprises more than two phosphatase inhibitors.

62. The tube of claim 60, wherein the tube further comprises an anticoagulant spray-dried onto at least a portion of the interior wall.

63. A kit for collecting and storing a biological sample for subsequent testing, comprising:
a primary collection tube having a separator element therein; and
a secondary tube;
wherein the primary collection tube and the secondary tube contain one or more protein stabilizing agents, the agents comprising at least one phosphatase inhibitor.

64. The kit of claim 63, wherein the one or more stabilizing agents are in a form selected from the group consisting of a solution, suspension or other liquid, a pellet, a tablet, a capsule, a spray-dried material, a freeze-dried material, a powder, a particle, a gel, crystals or a lyophilized material.

65. The kit of claim 64, wherein the one or more stabilizing agents are lyophilized.

66. The kit of claim 63, wherein the separator element is a mechanical separating element.

67. The kit of claim 66, wherein the mechanical separating element is at least partially coated with the one or more stabilizing agents.

68. The kit of claim 67, wherein the mechanical separating element is substantially inert with respect to the one or more stabilizing agents.

69. The kit of claim 63, wherein the separator element is a gel.

70. The kit of claim 69, wherein the gel is physically separated from the stabilizing agent.

71. The kit of claim 63, wherein the stabilizing agents further comprise at least one protease inhibitor.

72. The kit of claim 63, wherein the primary or secondary tube or both further contains a carrier media.

73. The kit of claim 63, wherein the primary or secondary tube or both further contain a stabilizing media.

74. The kit of claim 73, wherein the stabilizing media is trehalose.

75. The kit of claim 63, wherein the primary or secondary tube or both further contains at least one antioxidant.

76. The kit of claim 63, wherein the primary or secondary tube or both further contain at least one reducing agent.

77. The kit of claim 63, wherein the primary or secondary tube or both further contain at least one buffering agent.

78. The kit of claim 63, further comprising a tube-to-tube transfer device.

79. The kit of claim 78, wherein the second tube is maintained at a pressure to draw the sample from the first tube through the tube-to-tube transfer device and into the second tube.

80. A method of stabilizing a biological sample, comprising:
providing a sample collection container; and
disposing the biological sample into the collection container such that the sample is contacted with a protein stabilizing agent, the agent comprising at least one phosphatase inhibitor.

81. The method of claim 80, wherein the sample collection container includes the stabilizing agent before collecting the biological sample.

82. The method of claim 80, wherein the disposing of the biological sample into the container and the contacting of the sample with the stabilizing agent are performed in the same collection container.

83. The method of claim 82, wherein the collection container is evacuated and has a predetermined internal pressure sufficient to draw a predetermined volume of the sample into the collection container.

84. The method of claim 80, wherein the stabilizing agent is in a form selected from the group consisting of a solution, suspension or other liquid, a pellet, a tablet, a capsule, a spray-dried material, a freeze-dried material, a powder, a particle, a gel, crystals and a lyophilized material.

85. The method of claim 84, wherein the stabilizing agent is lyophilized.

86. The method of claim 80, wherein the collection container is selected from the group consisting of tubes, closed system blood collection devices, collection bags, syringes, microtiter plates, multi-well collection devices, flasks, spinner flasks, roller bottles and vials.

87. The method of claim 86, wherein the collection container is a tube.

88. The method of claim 87, wherein the collection container includes a separating member.

89. The method of claim 88, wherein the separating member is at least partially coated with the stabilizing agent.

90. The method of claim 88, wherein the separating member is a mechanical separating element.

91. The method of claim 90, wherein the mechanical separating element is inert with respect to the stabilizing agent.

92. The method of claim 88, wherein the separating member is a gel.

93. The method of claim 80, wherein the stabilizing agent further comprises at least one protease inhibitor.

94. The method of claim 80, wherein the at least one phosphatase inhibitor inhibits at least one phosphatase selected from the group consisting of serine phosphatase, threonine phosphatase, and tyrosine phosphatase.

95. The method of claim 80, wherein the stabilizing agent comprises more than two phosphatase inhibitors.

96. The method of claim 80, wherein the biological sample is selected from the group consisting of whole blood or a component thereof, red blood cell concentrates, platelet concentrates, leukocyte concentrates, plasma, serum, urine, bone marrow apirates, cerebral spinal fluid, tissue, cells, feces, saliva and oral secretions, nasal secretions and lymphatic fluid.

97. The method of claim 96, wherein the biological sample is whole blood.

98. The method of claim 97, wherein the whole blood is collected from a patient directly into the collection container.

99. The method of claim 98, wherein the collection container includes the stabilizing agent before the blood is collected from the patient.

100. A method for making a collection container for collecting a biological sample, comprising:
providing a collection container;
disposing stabilizing agent comprising at least one phosphatase inhibitor into the container;
lyophilizing the stabilizing agent;
evacuating and sealing the container; and
sterilizing the container.

101. The method of claim 100, wherein the stabilizing agent comprises more than two phosphatase inhibitors.

102. The method of claim 100, wherein the at least one phosphatase inhibitor inhibits at least on phosphatase selected from the group consisting of serine phosphatases, threonine phosphatases, and tyrosine phosphatases.

103. The method of claim 102, wherein stabilizing agent further comprises a protease inhibitor.

104. The method of claim 100, wherein the collection container is a tube.

105. The method of claim 104, further comprising placing into the tube a separating member.

106. The method of claim 105, wherein the separating member is a mechanical separating element.

107. The method of claim 105, wherein the separating member is a gel.

108. The method of claim 104, further comprising disposing an anticoagulant into the tube.

109. The method of claim 108, wherein the anticoagulant is disposed into the tube by spray-drying.

110. The method of claim 108, wherein the anticoagulant comprises a salt of EDTA.

111. The method of claim 108, wherein the anticoagulant comprises heparin.

112. A process for protein isolation, comprising:
providing a container comprising a protein stabilizing agent comprising at least one phosphatase inhibitor;
adding a biological sample to the container; and
isolating the protein from the biological sample.

113. The process of claim 112, wherein the collection container is a tube.

114. The process of claim 113, further comprising a separating member disposed in the container.

115. The process of claim 114, wherein the separating member is a mechanical separating element.

116. The process of claim 115, wherein the mechanical separating element is at least partially coated with the at least one stabilizing agent.

117. The process of claim 114, wherein the separating member is a gel and gel separating member is physically separated from the stabilizing agent.

118. The process of claim 113, wherein the stabilizing agent further comprises at least one protease inhibitor.

119. The process of claim 113, wherein the tube is partially evacuated.

120. The process of claim 119, wherein the stabilizing agent is lyophilized.

121. The process of claim 120, wherein the tube further comprises an anticoagulant.

122. The process of claim 121, wherein the anticoagulant is spray-dried onto at least a portion of an interior wall of the tube.

123. The process of claim 121, wherein the anticoagulant comprises a salt of EDTA.

124. The process of claim 121, wherein the anticoagulant comprises heparin.

125. The process of claim 112, wherein the at least one phosphatase inhibitor inhibits at least one phosphatase selected from the group consisting of serine phosphatases, threonine phosphatases, and tyrosine phosphatases.

126. The process of claim 112, wherein the stabilizing agent comprises more than two phosphatase inhibitors.
